(19) United States
(12) Patent Application Publication
(10) Pub. No.: US 2010/0234930 A1
(43) Pub. Date: Sep. 16, 2010

(54) IMPLANTABLE MEDICAL DEVICE LEAD ELECTRODE WITH CONSISTENT PORE SIZE STRUCTURE

(76) Inventors: Brendan E. Koop, Coon Rapids, MN (US); Peter C. Hall, Andover, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/704,811

(22) Filed: Feb. 12, 2010

Related U.S. Application Data
(60) Provisional application No. 61/159,130, filed on Mar. 11, 2009.

(51) Int. Cl. A61N 1/05; H01R 43/00

(52) U.S. Cl. 607/119; 29/874

(57) ABSTRACT

A method for making an implantable electrode for a cardiac lead includes forming a template including a plurality of features having substantially similar feature dimensions is formed. The template defines a shape corresponding to a shape of the implantable electrode. A layer of conductive material is then deposited on the template such that the conductive material shapes to the plurality of features to define an array of electrode pores having substantially similar pore dimensions in the layer of conductive material. The template is then removed from the layer of conductive material.

### CROSS-REFERENCE TO RELATED APPLICATION

This application claims priority to Provisional Application No. 61/159,130, filed Mar. 11, 2009, which is herein incorporated by reference in its entirety.

### TECHNICAL FIELD

The present invention relates to implantable medical devices. More particularly, the present invention relates to medical device electrodes with pores having substantially similar pore diameters.

### BACKGROUND

Cardiac pacing leads are well known and widely employed for carrying pulse stimulation signals to the heart from a battery operated pacemaker, or other pulse generating means, as well as for monitoring electrical activity of the heart from a location outside of the body. Electrodes are also used to stimulate the heart in an effort to mitigate bradycardia or terminate tachycardia or other arrhythmias. In all of these applications, it is highly desirable to optimize electrical performance characteristics of the electrode/tissue interface. Such characteristics include minimizing the threshold voltage necessary to depolarize adjacent cells, maximizing the electrical pacing impedance to prolong battery life, and minimizing sensing impedance to detect intrinsic signals.

Pacing (or stimulation) threshold is a measurement of the electrical energy required for a pulse to initiate a cardiac depolarization. The pacing threshold may rise after the development of a fibrous capsule around the electrode tip, which occurs over a period of time after implantation. The thickness of the fibrous capsule is generally dependent upon the mechanical characteristics of the distal end of the lead (i.e., stiff or flexible), the geometry of the electrode tip, the microstructure of the electrode tip, and the biocompatibility of the electrode and other device materials. In addition, the constant beating of the heart can cause the electrode to pound and rub against the surrounding tissue, causing irritation and a subsequent inflammatory response, eventually resulting in a larger fibrotic tissue capsule.

In a pacemaker electrode, minimal tissue reaction is desired around the tip, but firm intimate attachment of the electrode to the tissue is essential to minimize any electrode movement. A porous electrode tip with a tissue entrapping structure allows rapid fibrous tissue growth into a hollow area or cavity in the electrode tip to facilitate and enhance attachment of the electrode to the heart and increase biocompatibility. A reduced electrode dislodgement rate is also expected as a result of such tissue in-growth. A further aspect is selection of the average pore size, which must accommodate economical construction techniques, overall dimensional tolerances, and tissue response constraints. Tissue in-growth may assist in anchoring the electrode in place and increasing the contact surface area between the electrode and the tissue.

### SUMMARY

Discussed herein are various components for implantable medical electrical leads comprising an array of substantially similarly dimensioned pores, as well as medical electrical leads including such components.

In Example 1, a method for making an implantable electrode for a cardiac lead includes forming a template including a plurality of features having substantially similar feature dimensions. The template defines a shape corresponding to a shape of the implantable electrode. A layer of conductive material is then deposited on the template such that the conductive material shapes to the plurality of features to define an array of electrode pores having substantially similar pore dimensions in the layer of conductive material. The template is then removed from the layer of conductive material.

In Example 2, the method according to Example 1, wherein the electrode pores have diameters in the range about 30 μm to about 40 μm.

In Example 3, the method according to either Example 1 or Example 2, and further compring securing the layer of conductive material including the electrode pores to a bulk conductive material, wherein the layer of conductive material is comprised of the same material as the bulk conductive material.

In Example 4, the method according to any of Examples 1-3, wherein the forming step comprises forming a template including a plurality of closely-packed spheres having substantially similar dimensions.

In Example 5, the method according to any of Examples 1-4, wherein the depositing step comprises depositing a layer of conductive material on the template such that the conductive material that extends at least partially around the plurality of closely-packed spheres to define an array of electrode pores each having a diameter substantially similar to the spheres.

In Example 6, the method according to any of Examples 1-5, wherein the forming step comprises sintering the template.

In Example 7, the method according to any of Examples 1-6, wherein the shape of the implantable electrode is substantially hemispherical.

In Example 8, the method according to any of Examples 1-7, wherein the shape of the implantable electrode is substantially annular.

In Example 9, the method according to any of Examples 1-8, wherein depositing the layer of conductive material on the template comprises any of evaporating, sputtering, plating, and casting conductive material onto the template.

In Example 10, the method according to any of Examples 1-9, wherein the layer of conductive material is comprised of a metal.

In Example 11, the method according to any of Examples 1-10, wherein the template is comprised of a polymeric material or graphite.

In Example 12, a medical device lead includes a lead body having a conductor extending from a proximal end to a distal end. The proximal end is adapted to be connected to a pulse generator. The medical device lead also includes one or more electrodes having a layer of conductive material that defines an array of electrode pores having substantially similar dimensions. The layer of conductive material is electrically connected to the conductor.

In Example 13, the medical device lead according to Example 12, wherein the electrode pores are sized to minimize a thickness of collagen capsules that form on the electrode pores from tissue adjacent the one or more electrodes.

In Example 14, the medical device lead according to either Example 12 or Example 13, wherein the electrode pores have diameters in the range about 30 μm to about 40 μm.

In Example 15, the medical device lead according to any of Examples 12-14, wherein the template is comprised of sintered material.

In Example 16, the medical device lead according to any of Examples 12-15, wherein the template is comprised of a polymeric material or graphite.

In Example 17, the medical device lead according to any of Examples 12-16, wherein the layer of conductive material is comprised of a metal.

In Example 18, an implantable electrode for a cardiac lead includes a conductive layer that defines an array of electrode pores having substantially similar diameters in the range of about 30 μm to about 40 μm. The conductive layer is configured to communicate electrical signals between the cardiac lead and adjacent tissue.

In Example 19, the implantable electrode of Example 18, wherein the layer of conductive material is comprised of a metal.

In Example 20, the implantable electrode of either Example 18 or Example 19, wherein the electrode pores are substantially spherical.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic drawing of a cardiac rhythm management system including a pulse generator coupled to a lead including porous electrodes deployed in a patient's heart.

FIGS. 2A-2C are cross-section views of steps in a process for fabricating medical device lead electrodes having consistent pore sizes according to an embodiment of the present invention.

FIG. 3 is a cross-section of the distal end of a medical device lead including a porous metallic ring according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

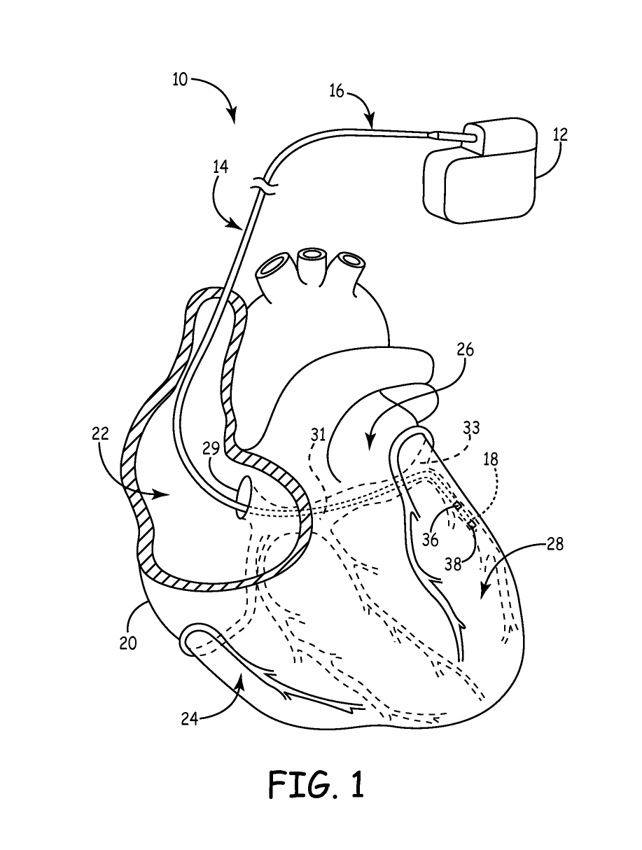

FIG. 1 is a schematic view of a cardiac rhythm management system 10 including an implantable medical device (IMD) 12 with a lead 14 having a proximal end 16 and a distal end 18. In one embodiment, the IMD 12 includes a pulse generator. The IMD 12 can be implanted subcutaneously within the body, typically at a location such as in the patient's chest or abdomen, although other implantation locations are possible. The proximal end 16 of the lead 14 can be coupled to or formed integrally with the IMD 12. The distal end 18 of the lead 14, in turn, can be implanted at a desired location in or near the heart 20.

As shown in FIG. 1, distal portions of lead 14 are disposed in a patient's heart 20, which includes a right atrium 22, a right ventricle 24, a left atrium 26, and a left ventricle 28. In the embodiment illustrated in FIG. 1, the distal end 18 of the lead 14 is transvenously guided through the right atrium 22, through the coronary sinus ostium 29, and into a branch of the coronary sinus 31 or the great cardiac vein 33. The illustrated position of the lead 14 can be used for sensing or for delivering pacing and/or defibrillation energy to the left side of the heart 20, or to treat arrhythmias or other cardiac disorders requiring therapy delivered to the left side of the heart 20. Additionally, it will be appreciated that the lead 14 can also be used to provide treatment in other regions of the heart 20 (e.g., the right ventricle 24).

Although the illustrative embodiment depicts only a single implanted lead 14, it should be understood that multiple leads can be utilized so as to electrically stimulate other areas of the heart 20. In some embodiments, for example, the distal end of a second lead (not shown) may be implanted in the right atrium 22, and/or the distal end of a third lead (not shown) may be implanted in the right ventricle 24. Other types of leads such as epicardial leads may also be utilized in addition to, or in lieu of, the lead 14 depicted in FIG. 1.

During operation, the lead 14 can be configured to convey electrical signals between the IMD 12 and the heart 20. For example, in those embodiments where the IMD 12 is a pacemaker, the lead 14 can be utilized to deliver electrical stimuli for pacing the heart 20. In those embodiments where the IMD 12 is an implantable cardiac defibrillator, the lead 14 can be utilized to deliver electric shocks to the heart 20 in response to an event such as a heart attack or arrhythmia. In some embodiments, the IMD 12 includes both pacing and defibrillation capabilities.

In the embodiment shown, the lead 14 includes ring electrode 36 and tip electrode 38 at distal end 18. The ring electrode 36 and the tip electrode 38 are connected to one or more conductors that extend through the lead body from the IMD 12 to the distal end 18. The ring electrode 36 and/or the tip electrode 38 may be configured to deliver therapeutic electrical signals generated by the IMD 12 to adjacent tissue in the heart 20. The ring electrode 36 and/or the tip electrode 38 may also be configured to sense activity in the heart 20, and provide electrical signals related to the sensed activity to the IMD 12.

According to the present invention, the ring electrode 36 and/or tip electrode 38 include a plurality of pores formed in the conductive electrode material that have substantially similar dimensions. The porous electrodes 36 and/or 38 promote tissue growth into the pores, thereby tethering the lead 14 to the adjacent tissue. In addition, the pores are sized to minimize the collagen capsule thickness of the ingrown tissue, thus minimizing the pacing threshold voltage needed to depolarize the tissue. In some embodiments, the pores have diameters in the range of about 30 μm to about 40 μm. The diameter refers to an average distance between two points across a pore.

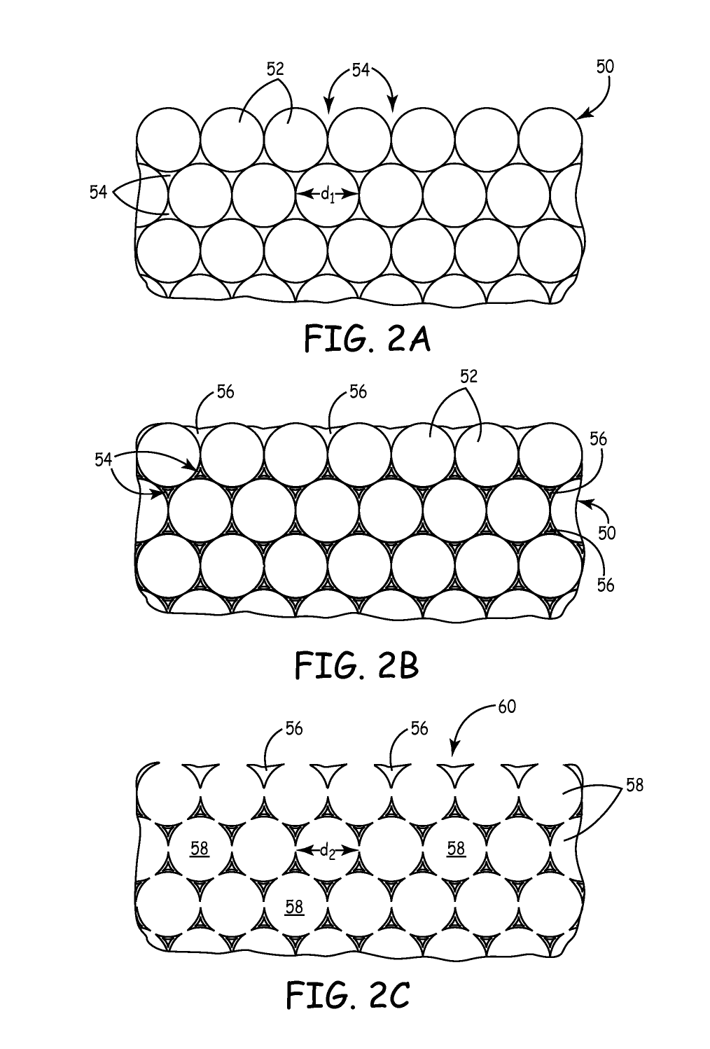

FIGS. 2A-2C are cross-section views of steps in a process for fabricating a porous electrode having a consistent pore size, according to an embodiment of the present invention. The process described in FIGS. 2A-2C may be employed to produce at least portions of either or both of the ring electrode 36 and/or the tip electrode 38. In addition, the process described may be used to produce at least portions of additional ring electrodes 36 or other types of electrodes not specifically shown FIG. 1. In FIG. 2A, a portion of a template 50 is shown including a plurality of closely-packed spheres 52 with a plurality of voids 54 between adjacent spheres 52. The spheres 52 have substantially similar diameters d₁. In some embodiments, the diameter d₁ is in the range of about 30 μm to about 40 μm. The spheres 52 may be comprised of a polymeric material, such as poly methyl methacrylate (PMMA). The spheres 50 may alternatively be comprised of graphite. The template 50 also includes voids 54 in the interior of the template 50 to form a network of interconnected voids scattered through the template 50.

The template 50 may be formed in a variety of ways. In one approach, beads of polymeric material are passed through one or more sieves to collect only beads having a certain desired size or diameter. The size of the beads collected is chosen based on the preferred pore size for the porous electrode. The appropriately sized beads are then shaken in a mold or other structure having a shape corresponding to the shape of the electrode. Shaking the beads causes the beads to closely pack into the mold. The beads are then connected together, such as by fusing the beads together with a sintering process, to produce the template 50. One suitable approach to fabricating the template 50 in this manner is described in U.S. patent application Ser. No. 10/595,233, entitled “Novel Porous Biomaterial,” which is hereby incorporated by reference in its entirety.

The voids 54 of the template 50 are shown in FIG. 2A having uniform depths, but it will be appreciated that the depths of the voids 54 may vary across the template 50. In addition, while the voids are shown regularly spaced apart, interconnected by the spheres 52 extending between the voids 54, it will be understood that the spacing between the pores may be less or more regular than what is illustrated. In other words, while the spheres 52 are shown having substantially identical diameters d₁, the diameters of the spheres 52 may vary slightly across the template. It will also be appreciated that in actual implementation, the voids 54 may be randomly scattered throughout the template 50.

After the template 50 is formed, a conductive layer 56 is deposited onto the template 50, as shown in FIG. 2B. The conductive layer 56 extends around the spheres 52 and into the voids 54. Consequently, the conductive layer 56 at least partially surrounds the spheres 52 throughout the template 50 when the conductive layer 56 is deposited on the template 50.

The conductive layer 56 may be comprised of a material suitable for an implantable medical device lead electrode, such as platinum (Pt), platinum-iridium (PtIr), titanium (Ti), nickel-titanium (NiTi), tantalum (Ta), MP35N alloy, or stainless steel. The conductive layer 56 covers at least a portion of the surface of the template 50 and infiltrates into the voids 54 to coat portions of the template 50 that define the voids 54. In some embodiments, the conductive layer 56 does not completely coat the outer surface of the template 50. For example, in the embodiment shown, the conductive layer 56 is deposited into the voids 54 but not on portions of the spheres 52 along the outer surface of the template 50.

The conductive layer 56 may be formed on the template 50 in a variety of ways. For example, the conductive layer 56 may be evaporated, sputtered, or plated on the template 50. As another example, the conductive layer 56 may also be deposited onto the template 50 using powder metallurgy techniques. As a further example, in embodiments in which the template 50 is comprised of graphite, the conductive layer 56 may be cast onto the template 50. It will be appreciated that the preceding examples should not be construed as limiting, and any suitable deposition technique may be used.

After the conductive layer 56 is deposited onto template 50, the template 50 may then be removed, as shown in FIG. 2C. The template 50 may be removed either during processing of the conductive layer 56, or during a separate process. For example, the template 50 may be evaporated, vaporized, or sublimated by subjecting the template 50 to certain temperature or pressure conditions.

When the template 50 is removed from the conductive layer 56, the conductive layer 56 defines an array of electrode pores 58. The pattern and size of the electrode pores 58 defined by the conductive layer 56 substantially matches the pattern of spheres 52 in the template 50. In the embodiment shown, the electrode pores 58 are substantially spherical. However, it will be appreciated that electrode pores 58 may be formed into other shapes (e.g., ellipsoidal) using differently shaped elements in the template 50.

Each electrode pore 58 has a diameter d₂. In order to form electrode pores 58 having the desired diameters d₂, the diameters d₁ of the spheres 52 are approximately equal to the desired diameter d₂. Thus, because the spheres 52 have substantially similar diameters, the diameters d₂ of the electrode pores 58 are also substantially similar to each other. In some embodiments, the diameters of the electrode pores 58 are within about 20% of the mean diameter of the electrode pores 58.

The electrode including electrode pores 58 promotes tissue in-growth to secure the lead 14 relative to the adjacent tissue of the heart 20, thereby lowering the likelihood of dislodgement of the lead 14. In addition, the electrode pores 58 are sized substantially similar with dimensions to minimize the thickness of collagen capsules of ingrown tissue. Collagen capsules with reduced thickness allow a lower voltage to be used to depolarize surrounding myocardial tissue. A minimum thickness of fibrous encapsulation occurs when the diameter of the electrode pores 58 is about 35 μm. Thus, in some embodiments, the diameter d₂ of the electrode pores 58 is in the range of about 30 μm to about 40 μm.

When the template 50 is removed, the remaining conductive layer 56, which has the shape of the electrode being fabricated, may be incorporated into the lead 14. A layer of bulk conductive material may also be secured to the conductive layer 56 opposite tissue-confronting surface 60 to provide added conductor thickness to the electrode assembly.

FIGS. 2A-2C illustrate formation of an electrode using a positive template 50 to form the conductive layer 56 with electrode pores 58. That is, the spheres 52 defined the location of the electrode pores 58. In an alternative embodiment of the present invention, a negative template may be employed to construct an electrode (e.g., ring electrode 36 and/or tip electrode 38), in which voids in the template structure define the location of the electrode pores and the solid portions of the template shape the solid portions of the electrode. The negative template may be comprised of materials similar to those described above with regard to FIG. 2A. In addition, the negative template may be formed using the molding and sintering technique described above. The negative template may also be formed by creating a porous polymer foam with tightly controlled pore size and interconnected pores. The conductive layer, which may be comprised of materials similar to conductive layer 56, may be deposited over the negative template, and the negative template may be subsequently removed to provide a conductive layer that has the shape of the electrode being fabricated. In an alternative embodiment, the negative template itself is formed of a conductive material in the shape of the electrode and having a tightly controlled pore size.

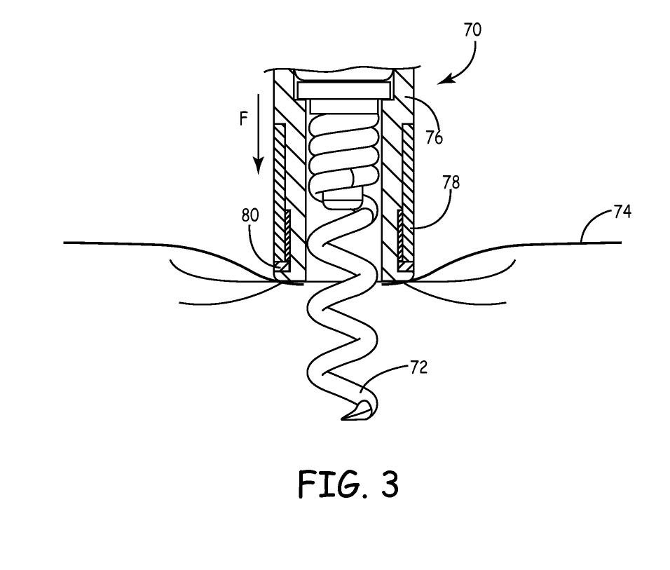

While the structures including consistent pore sizes have been described with regard to medical device lead electrodes, other types of metallic structures on medical device leads may also be formed with consistent pore sizes to encourage tissue in-growth. For example, FIG. 3 is a cross-section view of the distal end of a medical device lead 70 including a fixation helix 72 engaged with tissue 74, such as from the heart 20. The fixation helix 72 causes the lead 70 to engage the tissue 74 with a downward force F. Over time, this force F can result in perforation of the tissue 74, and cause the distal end of the lead 70 to pass through the surface of the tissue 74 and into the underlying organ. To prevent this, a metal element 76 including consistently sized pores may be formed at the distal end of the lead 70. The metal element 76 may be formed using any of the techniques described above. In some embodiments, the metal element 76 includes pores having an average diameter of about 35 μm. In the embodiment shown, metal element 76 extends behind steroid collar 78 and fluoroscopic marker 80 and confronts the tissue 74 at the distal end. When implanted, the tissue 74 grows into the pores of the metal element 76 and tethers the lead 70 at the location of implantation, thereby preventing perforation of the tissue 74 by the lead 70.

In summary, the present invention relates to an implantable electrode and a method for making an implantable electrode for a cardiac lead. A template including a plurality of features having substantially similar feature dimensions is formed. The template defines a shape corresponding to a shape of the implantable electrode. A layer of conductive material is then deposited on the template such that the conductive material shapes to the plurality of features to define an array of electrode pores having substantially similar pore dimensions in the layer of conductive material. The template is then removed from the layer of conductive material. The electrode pores may be sized to minimize the thickness of collagen capsules of ingrown tissue, which minimizes the threshold voltage of the ingrown tissue. In some embodiments, the pores have diameters in the range of about 30 μm to about 40 μm. In addition, the tissue in-growth secures the lead relative to the adjacent tissue, thereby lowering the likelihood of dislodgement of the cardiac lead.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof. For example, while the present invention has been described with regard to cardiac leads, electrodes having consistent pore sizes as described may also be used in other types of leads, such as neurological and spinal leads.

What is claimed is:

1. A method for making an implantable electrode for a cardiac lead, the method comprising:
forming a template including a plurality of features having substantially similar feature dimensions, wherein the template defines a shape corresponding to a shape of the implantable electrode;
depositing a layer of conductive material on the template such that the conductive material shapes to the plurality of features to define an array of electrode pores in the layer of conductive material, wherein the electrode pores have substantially similar pore dimensions; and
removing the template from the layer of conductive material.

2. The method of claim 1, wherein the electrode pores have diameters in the range about 30 μm to about 40 μm.

3. The method of claim 1, and further comprising:
securing the layer of conductive material including the electrode pores to a bulk conductive material, wherein the layer of conductive material is comprised of the same material as the bulk conductive material.

4. The method of claim 1, wherein the forming step comprises forming a template including a plurality of closely-packed spheres having substantially similar dimensions.

5. The method of claim 4, wherein the depositing step comprises depositing a layer of conductive material on the template such that the conductive material that extends at least partially around the plurality of closely-packed spheres to define an array of electrode pores each having a diameter substantially similar to the spheres.

6. The method of claim 1, wherein the forming step comprises sintering the template.

7. The method of claim 1, wherein the shape of the implantable electrode is substantially hemispherical.

8. The method of claim 1, wherein the shape of the implantable electrode is substantially annular.

9. The method of claim 1, wherein depositing the layer of conductive material on the template comprises any of evaporating, sputtering, plating, and casting conductive material onto the template.

10. The method of claim 1, wherein the layer of conductive material is comprised of a metal.

11. The method of claim 1, wherein the template is comprised of a polymeric material or graphite.

12. A medical device lead comprising:
a lead body including a conductor extending from a proximal end to a distal end, wherein the proximal end is adapted to be connected to a pulse generator; and
one or more electrodes including a layer of conductive material defining an array of electrode pores having substantially equal diameters, wherein the layer of conductive material is electrically connected to the conductor.

13. The medical device lead of claim 12, wherein the electrode pores are sized to minimize a thickness of collagen capsules that form on the electrode pores from tissue adjacent the one or more electrodes.

14. The medical device lead of claim 12, wherein the electrode pores have diameters in the range about 30 μm to about 40 μm.

15. The medical device lead of claim 12, wherein the template is comprised of sintered material.

16. The medical device lead of claim 12, wherein the template is comprised of a polymeric material or graphite.

17. The medical device lead of claim 12, wherein the layer of conductive material is comprised of a metal.

18. An implantable electrode for a cardiac lead, the implantable electrode comprising a conductive layer that defines an array of electrode pores having substantially similar diameters in the range of about 30 μm to about 40 μm, wherein the conductive layer is configured to communicate electrical signals between the cardiac lead and adjacent tissue.

19. The implantable electrode of claim 18, wherein the layer of conductive material is comprised of a metal.

20. The implantable electrode of claim 18, wherein the electrode pores are substantially spherical.
